(19) United States
(12) Patent Application Publication
(10) Pub. No.: US 2021/0346409 A1
(43) Pub. Date: Nov. 11, 2021

(54) COVID-19 THERAPEUTICS AND METHODS OF TREATMENT

(71) Applicant: Board of Supervisors of Louisiana State University and Agricultural and Mechanical College, Baton Rouge, LA (US)

(72) Inventors: Johnathan Steven ALEXANDER, Shreveport, LA (US); Christopher KEVIL, Shreveport, LA (US); Mansoureh BARZEGAR, Lafayette, LA (US); Ana Maria DRAGOI, Shreveport, LA (US)

(73) Assignee: Board of Supervisors of Louisiana State University and Agricultural and Mechanical College, Baton Rouge, LA (US)

(21) Appl. No.: 17/315,249

(22) Filed: May 7, 2021

Related U.S. Application Data
(60) Provisional application No. 63/021,368, filed on May 7, 2020.

(51) Int. Cl. A61K 31/655; A61K 31/397; A61K 31/4184; A61K 31/41; A61K 31/366; A61K 31/22; A61K 31/405; A61K 31/505; A61K 31/4418; A61K 31/40; A61K 31/47; A61K 31/4245; A61K 31/4178

(52) U.S. Cl. CPC A61K 31/655; A61K 31/397; A61K 31/4184; A61K 31/41; A61K 31/366; A61K 31/22; A61K 31/4178; A61K 31/505; A61K 31/4418; A61K 31/40; A61K 31/47; A61K 31/4245; A61K 31/405

(57) ABSTRACT

Therapeutics and methods of treating COVID-19 in a patient in need thereof comprising administering a pharmaceutical composition containing a pharmaceutically effective dose of a therapeutic, wherein the therapeutic contains both an ACE2 externalizer and one or more ACE2 internalization preventors. Methods of treatment and pharmaceutical compositions comprising an ACE2 externalizer and one or more ACE2 internalization preventors.

### CROSS REFERENCE TO RELATED APPLICATIONS/PRIORITY

The present invention claims priority to United States Provisional Patent Application No. 63/021,368 filed May 7, 2020, which is incorporated by reference into the present disclosure as if fully restated herein. Any conflict between the any incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

The severe acute respiratory syndrome coronavirus 2 (SARS-CoV-2), which causes COVID-19 (coronavirus disease 2019), has infected over 157,000,000 people and killed over 3,000,000 people worldwide in a little over one year, with the number continuing to rise and no cure yet available. Vaccines have been developed for some strains of the virus, but newly mutated strains continue to appear. For these reasons there is a pressing but seemingly irresolvable need to discover a successful treatment for COVID-19.

### SUMMARY

Wherefore, it is an object of the present invention to overcome the above-mentioned shortcomings and drawbacks associated with the current technology.

The presently disclosed invention is related to therapeutics and methods of treating COVID-19 in a patient in need thereof comprising administering a pharmaceutical composition containing a pharmaceutically effective dose of a therapeutic, wherein the therapeutic contains both an ACE2 externalizer and one or more ACE2 internalization preventors. According to a further embodiment the ACE2 externalizer is one of diminazene, diminazene aceturate, or a pharmaceutically acceptable salt, solvate, ester, amide, clathrate, stereoisomer, enantiomer, prodrug or analog thereof. According to a further embodiment the one or more ACE2 internalization preventor is one or more of a cholesterol binding drug, a cholesterol synthesis inhibitor, and an angiotensin type 1 receptor antagonist. According to a further embodiment the cholesterol binding drug is ezetimibe or a pharmaceutically acceptable salt, solvate, ester, amide, clathrate, stereoisomer, enantiomer, prodrug or analog thereof. According to a further embodiment the cholesterol synthesis inhibitor is one of atorvastati, cerivastatin, fluvastatin, lovastatin, mevastatin, pitavastatin, pravastatin, rosuvastatin, and simvastatin, or a pharmaceutically acceptable salt, solvate, ester, amide, clathrate, stereoisomer, enantiomer, prodrug or analog thereof. According to a further embodiment the angiotensin type 1 receptor antagonist is one of azilsartan, candesartan, eprosartan, irbesartan, telmisartan, valsartan, losartan, olmesartan, entresto, byvalson and fimasartan or a pharmaceutically acceptable salt, solvate, ester, amide, clathrate, stereoisomer, enantiomer, prodrug or analog thereof. According to a further embodiment the one or more ACE2 internalization preventor includes at least one of each of a cholesterol binding drug, a cholesterol synthesis inhibitor, and an angiotensin type 1 receptor antagonist.

The presently disclosed invention is further related to methods of treatment and pharmaceutical compositions comprising an ACE2 externalizer and one or more ACE2 internalization preventors. According to a further embodiment the ACE2 externalizer is one of diminazene, diminazene aceturate, or a pharmaceutically acceptable salt, solvate, ester, amide, clathrate, stereoisomer, enantiomer, prodrug or analog thereof. According to a further embodiment the one or more ACE2 internalization preventor is one or more of a cholesterol binding drug, a cholesterol synthesis inhibitor, and an angiotensin type 1 receptor antagonist. According to a further embodiment the cholesterol binding drug is ezetimibe or a pharmaceutically acceptable salt, solvate, ester, amide, clathrate, stereoisomer, enantiomer, prodrug or analog thereof. According to a further embodiment the cholesterol synthesis inhibitor is one of atorvastati, cerivastatin, fluvastatin, lovastatin, mevastatin, pitavastatin, pravastatin, rosuvastatin, and simvastatin, or a pharmaceutically acceptable salt, solvate, ester, amide, clathrate, stereoisomer, enantiomer, prodrug or analog thereof. According to a further embodiment the angiotensin type 1 receptor antagonist is one of azilsartan, candesartan, eprosartan, irbesartan, telmisartan, valsartan, losartan, olmesartan, entresto, byvalson and fimasartan or a pharmaceutically acceptable salt, solvate, ester, amide, clathrate, stereoisomer, enantiomer, prodrug or analog thereof. According to a further embodiment the one or more ACE2 internalization preventor includes at least one of each of a cholesterol binding drug, a cholesterol synthesis inhibitor, and an angiotensin type 1 receptor antagonist.

The presently disclosed invention is further related to therapeutics and methods of treating COVID-19 in a patient in need thereof comprising administering a pharmaceutically effective dose of a therapeutic, wherein the therapeutic contains an ACE2 externalizer. According to a further embodiment the ACE2 externalizer is one of diminazene, diminazene aceturate, or a pharmaceutically acceptable salt, solvate, ester, amide, clathrate, stereoisomer, enantiomer, prodrug or analog thereof.

The present invention relates to pharmaceutical compositions of a therapeutic (e.g., an ACE2 externalizer or an ACE2 externalizer and one or more ACE2 internalization preventors), or a pharmaceutically acceptable salts, solvates, esters, amides, clathrates, stereoisomers, enantiomers, prodrugs or analogs thereof, and use of these compositions for the treatment of COVID-19.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-100 μM (e.g., between 1.0 μM-25 μM).

In some embodiments, the peak plasma concentration of the therapeutic is maintained for up to 14 hours. In other embodiments, the peak plasma concentration of the therapeutic is maintained for up to 1 hour.

In some embodiments, the condition is COVID-19.

In certain embodiments, the COVID-19 is mild to moderate COVID-19.

In further embodiments, the COVID-19 is moderate to severe COVID-19.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the COVID-19.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., an ACE2 externalizer and an ACE2 internalization preventor), or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., exposure to the SARS-CoV-2 virus, subsequent infection, and/or COVID-19). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a graph showing diminazene aceturate concentration-dependently causes externalization of ACE2 and increases ACE2-mediated binding and signal of VSV virus-expressing COVID-spike protein in target cells.

FIG. 2 is a graph of externalization of ACE2 on cell surface after treatment with diminazene. The inventors cultured human brain endothelial cells to confluency and exposed these cells to 10 mM Diminazene for 3 hours after which they were fixed in 3.7% phosphate buffered formaldehyde for 30 mins. Cells were then incubated in 1:250 rabbit antibhuman ACE2 (overnite) followed by washes and then goat anti rabbit horseradish peroxidase (1 h). After color reaction, and increased average signal in treated monolayers consistent with externalization of ACE2 on the cell surface was shown (n=4).

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(asecond number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In addition, the invention does not require that all the advantageous features and all the advantages of any of the embodiments need to be incorporated into every embodiment of the invention.

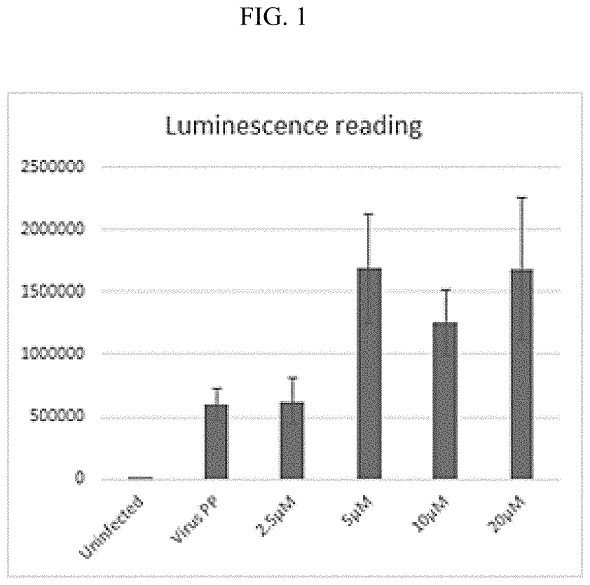

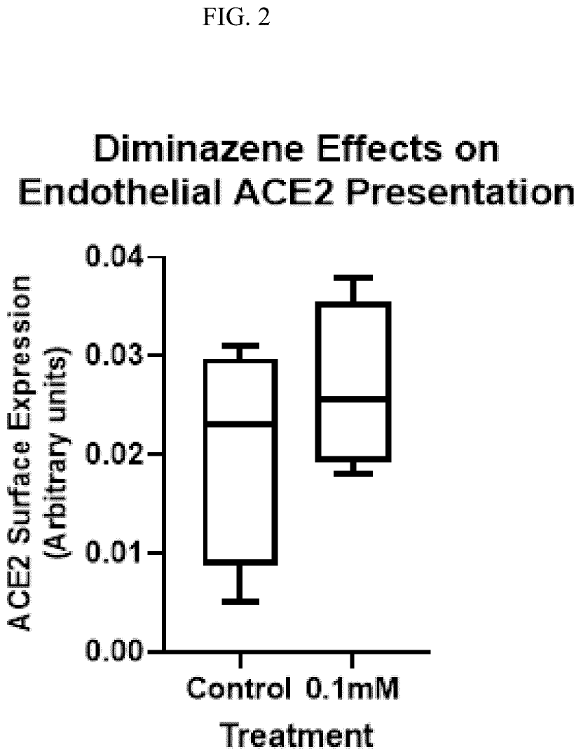

Turning now to FIGS. 1 and 2, a brief description concerning the various components of the present invention will now be briefly discussed.

A first embodiment of the disclosed invention relates to treatment of COVID-19 symptoms via the administration of an ACE2 externalizer. A second embodiment of the disclosed invention relates to a treatment of COVID-19 symptoms and locking down of further cell penetration of SARS-CoV-2 viruses, and allowing the body to fight the disease without the further recruitment of new viruses, via the administration of an ACE2 externalizer and one or more ACE2 internalization preventors.

According to the first embodiment of the invention, Berenil aceturate (Diminazene), described as an angiotensin converting enzyme-2 (ACE2) activator, but discovered by the inventors to actually be a ACE2 externalizer, is administered to treat COVID-19 infection associated ‘cytokine storm’ and ventilation-perfusion mismatch. The COVID-19 virus uses ACE2 as the receptor to penetrate into target cells—pulmonary endothelial cells and alveolar epithelial cells. SARS-CoV-2 cell entry depends on ACE2 and TMPRSS2. Infection with COVID-19 will depress the normal capacity of these cells to process Angiotensin II, a potent inflammatory vasoconstrictor, into Angiotensin 1-7, an anti-inflammatory vasodilator. The inhibition of ACE2 will lead to elevated levels of Ang2 due to a lack of the ability to clear this signal. The ACE2 externalizer diminazene, however, will move ACE2 to the surface of the cell and “rescue” activity of the ACE2 in these cells. Diminazene has been shown to specifically suppress the complement of inflammatory cytokines which are activated in COVID-19 including IL-6, TNF-a, IL-12 and IFN-g. The inventors know that treatment with diminazene reduces serum pro-inflammatory cytokine levels in T. congolense-infected mice.

ACE2 inhibition by COVID-19 will lead to a feedback/back up where AngII is not forward catabolized to Ang1-9, Ang1-7, Alamandine leading to withdrawal of vasoconstriction (and bronchoconstriction), keys symptoms in COVID-19 pathophysiology. Treatment with an ACE2 externalizer, such as diminazene salt, either aceturate or other compounding, to externalize ACE2 enables the catalytic capacity of ACE-2 during coronavirus infection mediated pulmonary pathophysiology. The ACE2 externalizer may be administered as an aerosolized form, or given intravenously or orally, for example. Because the coronavirus SARS-CoV-2 uses the cell surface receptor ACE-2 to bind and enter cells, the downregulation of this receptor during viral entry leads to lower levels of Angiotensin 1-7 which is a potent vasodilator. Furthermore, the processing of Angiotensin-2 to Angiotensin 1-7, a normal part of the catabolic processing of Angiotensin-2 leads to excessive Angiotensin-2, which drives vasoconstriction in the pulmonary circulation and other vascular beds. Similarly, ACE-2 in the alveolar epithelial cells may be needed to control airway reactivity through a similar process involving the formation of Ang-1-7 which controls airway remodeling necessary for normal ventilation. The use of the ACE2 externalizer alone will mediate some of the symptoms of COVID-19 caused by the Sars-CoV-2 preventing the normal capacity of these infected cells to process Angiotensin II, into Angiotensin 1-7, treating the disease and allowing the body more time and capacity to fight the infection.

This embodiment discloses an ACE2 externalizer, such as diminazene, an FDA approved trypanosomal, to treat COVID-19 symptoms, thus enabling clinicians to provide a safe treatment which is shown to a) reduce cytokines and b) restore AngII/Ang1-7 balance which are both dysregulated in COVID-19 infection. This treatment may be used to treat individuals who are seen in intensive care units or at emergency departments for COVID-19 associated respiratory distress, particularly when sPO2 is compromised and elevated pulmonary resistance is encountered. This treatment, when given to individuals who are suffering from hypoxia and airway constriction, would be expected to experience relief by increasing ACE-2 activity and expression to enhance Ang1-7 abundance in the lung.

According to a second embodiment, the disclosed invention relates to the combined use of an ACE2 externalizer and an ACE2 internalization preventor as a treatment and/or cure for COVID 19 infection. This therapy involves the combined use of at least two different classes of agents, one of which is a therapeutic that triggers the physical externalization of ACE 2 in target cells. These target cells could be in all endothelial cells (e.g., in the lung heart or brain for example, but not limited to these organs), epithelial cells in the lung or intestine or other cells in the body which express ACE 2 on their surface e.g., neurons, astrocytes or glial cells in the brain. Because ACE 2 is known to be the receptor for the COVID 19 virus, acute infection with this virus may lead to downregulation of the surface presented ACE 2 which is exploited by the COVID virus to gain entry into the cytoplasm. When diminazene is added to target cells susceptible to infection, it forces the externalization of ACE2 from internal stores, which increases the local activity of ACE2 in these cells. This effect explains the paradoxical increase in ACE2 activity, which has previously not been explained by increased catalytic activity with diminazene or increased protein expression. Diminazene induced ACE2 externalization can restore the normal balance of Angiotensin-2 to Angiotensin 1-7. Because Angiotensin-2 is a vasoconstrictor, pro-thrombotic and pro-inflammatory agent, the decrease in surface exposed ACE 2 produced by COVID virus leads to several pathologic phenomena in this type of infection, which includes vascular inflammation, intravascular coagulation and vasoconstriction in target organs particularly the heart lung and brain. When diminazene is used therapeutically, it will increase the ACE 2 activity in the bloodstream and in the lung compartments by forcing greater presentation of more active ACE2 in those compartments.

The inventors discovered that diminazene is able to induce ACE 2 externalization, a mechanism for diminazene in benefit in many different models. The inventors' research group has recognized that ACE2 provides significant and dramatic protection against ischemic stress.

The second part of this therapy of the second embodiment is to use ACE2 internalization preventors, such as agents that have been used to maintain normal blood cholesterol and blood pressure e.g. ezetimibe, angiotensin type 1 receptor antagonists, and statin cholesterol drugs, to trap the bound SARS-CoV-2 viruses on the surface of the cells, preventing the viruses from infiltrating the cells and replicating, and the trapped SARS-CoV-2 viruses subsequently degrading in a span of preferably 12 hours or less, through continued exposure to the harsh extra-cellular environment.

The use of drugs that modify cholesterol availability also suppresses endocytosis and can be used as the second half of this combination therapeutic approach. As well, angiotensin type 1 receptor antagonists have powerful effects against the induction of ACE2 internalization. By combining the use of an ACE2 externalizer, such as diminazene, plus one or more of an ACE2 internalization preventor such as 1) a cholesterol binding drug, such as ezetimibe, 2) a cholesterol synthesis inhibitors and/or 3) an angiotensin type 1 receptor antagonists, such as azilsartan (Edarbi), candesartan (Atacand), eprosartan (Teveten), irbesartan (Avapro), telmisartan (Micardis), valsartan (Diovan, Prexxartan), losartan (Cozaar), olmesartan (Benicar), entresto (sacubitril/valsartan), byvalson (nebivolol/valsartan), and fimasartan (Kanarb), ACE2 will be forced to the cell's surface and maintained it at this location, preventing internalization of the ACE2 bound SARS-CoV-2 viruses, while essentially normalizing vasoconstriction, coagulation and inflammation, biologic functions that can be dramatically adversely impacted by COVID-19. Similarly, this combination of therapeutics would produce beneficial effects in the pulmonary compartment by limiting viral penetration as well producing bronchodilation, vasodilation and suppressing local clotting processes.

Diminazene will preferably be administered such that a final circulating blood concentration of between 1 uM and 100 uM, more preferably 5 uM and 50 uM, and most preferably 10 uM is achieved. This concentration could be achieved by oral administering preferably up to 500 milligrams (e.g., in tablet form) taken one or more times daily preferably up to 40 milligrams per kilogram patient body weight.

Cholesterol synthesis inhibitors, such as statin-type drugs, (HMG CoA reductase inhibitor drugs lovastatin, pravastatin, etc.) could also be given preferably up to 100 milligrams per day to limit COVID19 infection and suppress its associated pathologies in the vascular and pulmonary spaces. Polypharmacy (combined use of drugs) which includes statins, plus angiotensin type 1 receptor antagonists, plus ezetimibe have been used to safely manage cardiovascular risks particularly in elderly or at-risk individuals; the use in COVID-19 as part of the second embodiment of this invention is similarly expected to have a good safety profile.

The dose of angiotensin type 1 receptor antagonists, such as losartan, would preferably be given such that a concentration of between 1 uM and 100 uM, more preferably 5 uM and 50 uM, and most preferably 10 uM is achieved. For losartan, a dose of 414 mg for a 100 kg human per day would achieve this effect and reach a concentration of 10 uM assuming complete biological distribution within tissues. The dose of cholesterol binding drug, such as ezetimibe, would preferably be given such that a concentration of between 1 uM and 100 uM, more preferably 5 uM and 50 uM, and most preferably 10 uM is achieved. For ezetimibe, a dose of 410 mg per day to would achieve this effect and reach a concentration of 10 uM in a 100 kg human assuming complete biological distribution within tissues. Similarly, cholesterol synthesis inhibitors may also be used in this manner to reduce endocytosis of ACE2, but should preferably not be used over a concentration of 20 mg/day as there is a potential risk for rhabdomyolysis.

The inventors discovered that diminazene is a drug which forces the externalization of ACE2, which is a vasoprotective and anti-coagulant surface, and which also happens to be the receptor for COVID19. The inventors are aware that angiotensin type 1 receptor antagonists prevent the internalization of ACE2. Similarly, ezetimibe and cholesterol synthesis inhibitors also represent classes of drugs which block endocytosis and limit the internalization of ACE2 forced to the external cell membrane by the action of diminazene. Maintaining patients on combination therapy with one or more ACE2 externalizers and one or more ACE2 internalization preventors, will promote at least two specific clinical benefits: 1) the beneficial balance of surface ACE2 will be restored, ACE2 being an anti-coagulant, anti-inflammatory and vasorelaxant, effects which are diminished or lost in COVID infection, 2) secondly, this combination of drugs will ‘lock out’ the SARS-CoV-2 virus against penetration into the cytoplasm of the cell, thereby preventing propagation and infection spread.

ACE2 externalizer: Diminazene is a ACE2 externalizer. This function of diamidine and may be formulated as its aceturate salt, diminazene aceturate, for example. Diminazene is also known as 4,4′-(1-Triazene-1,3-diyl) bis(benzenecarboximidamide), and has a chemical formula of C₁₄H₁₅N₇ and structural formula of

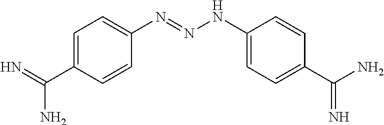 [SMILES: N=C(N)c1ccc(/N=N/Nc2ccc(C(=N)N)cc2)cc1]

ACE2 internalization preventor: The inventors have identified multiple ACE2 internalization preventors, as the following three classes of chemicals 1) cholesterol binding drugs, 2) cholesterol synthesis inhibitors, and 3) angiotensin type 1 receptor antagonists.

The following chemicals function to bind cholesterol and retain ACE2 at the surface of the cell: Ezetimibe.

Ezetimibe has the following structural formula:

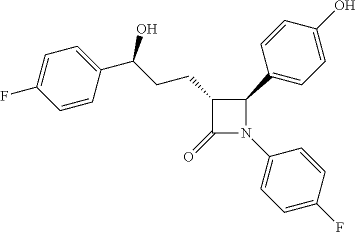 [SMILES: O=C1[C@H](CC[C@H](O)c2ccc(F)cc2)[C@@H](c2ccc(O)cc2)N1c1ccc(F)cc1]

The following chemicals function to inhibit cholesterol synthesis and retain ACE2 at the surface of the cell: atorvastati, cerivastatin, fluvastatin, lovastatin, mevastatin, pitavastatin, pravastatin, rosuvastatin, and simvastatin.

Atorvastatin has the following structural formula:

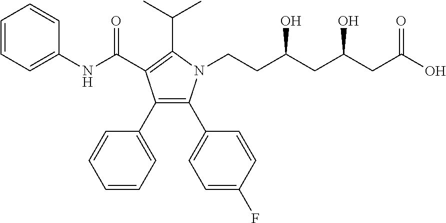 [SMILES: CC(C)c1c(C(=O)Nc2ccccc2)c(-c2ccccc2)c(-c2ccc(F)cc2)n1CC[C@@H](O)C[C@@H](O)CC(=O)O]

Cerivastatin has the following structural formula:

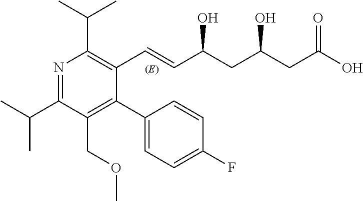 [SMILES: COCc1c(C(C)C)nc(C(C)C)c(/C=C/[C@@H](O)C[C@@H](O)CC(=O)O)c1-c1ccc(F)cc1]

Fluvastatin has the following structural formula:

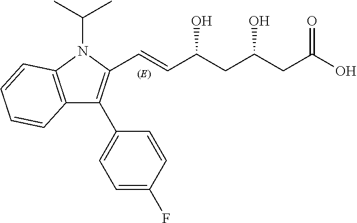 [SMILES: CC(C)n1c(/C=C/[C@H](O)C[C@H](O)CC(=O)O)c(-c2ccc(F)cc2)c2ccccc21]

Lovastatin has the following structural formula:

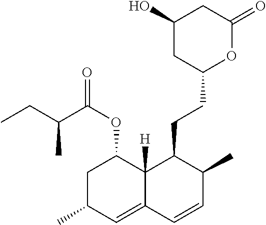 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

Mevastatin has the following structural formula:

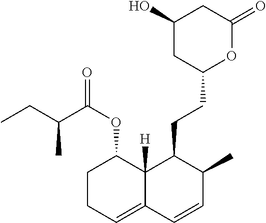 [SMILES: CC[C@H](C)C(=O)O[C@H]1CCC=C2C=C[C@H](C)[C@H](CC[C@@H]3C[C@@H](O)CC(=O)O3)[C@H]21]

Pitavastatin has the following structural formula:

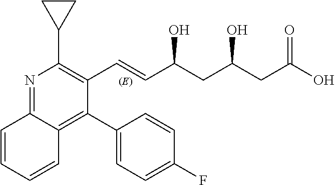 [SMILES: O=C(O)C[C@H](O)C[C@H](O)/C=C/c1c(C2CC2)nc2ccccc2c1-c1ccc(F)cc1]

Pravastatin has the following structural formula:

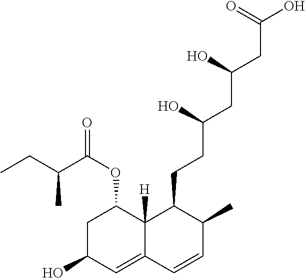 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

Rosuvastatin has the following structural formula:

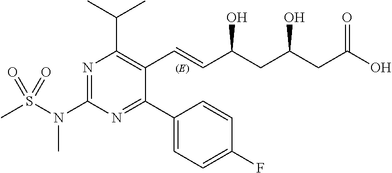 [SMILES: CC(C)c1nc(N(C)S(C)(=O)=O)nc(-c2ccc(F)cc2)c1/C=C/[C@@H](O)C[C@@H](O)CC(=O)O]

Simvastatin has the following structural formula:

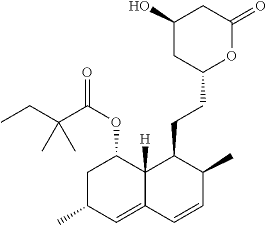 [SMILES: CCC(C)(C)C(=O)O[C@H]1C[C@@H](C)C=C2C=C[C@H](C)[C@H](CC[C@@H]3C[C@@H](O)CC(=O)O3)[C@H]21]

The following chemicals are antagonists for angiotensin type 1 receptor and retain ACE2 at the surface of the cell: azilsartan, candesartan, eprosartan, irbesartan, telmisartan, valsartan, losartan, olmesartan, entresto, byvalson and fimasartan.

Candesartan has the following structural formula:

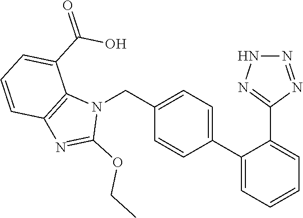 [SMILES: CCOc1nc2cccc(C(=O)O)c2n1Cc1ccc(-c2ccccc2-c2nn[nH]n2)cc1]

Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that cause ACE2 to externalize to the cell's surface (including diminazene) together with those that prevent ACE2 on the cell's surface from internalizing (including ezetimibe and candesartan).

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 5000 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The present methods for treating COVID-19s are carried out by administering a therapeutic for a time and in an amount sufficient to result in decreased hypoxia, cough, wheezing, confusion, rapid breathing, shortness of breath, and a normalization of heart rate, and preferably a time frame that allows for a sustained decrease in such symptoms.

The amount and frequency of administration of the compositions can vary depending on, for example, what is being administered, the state of the patient, and the manner of administration. In therapeutic applications, compositions can be administered to a patient suffering from COVID-19 in an amount sufficient to relieve or least partially relieve the symptoms of the COVID-19 and its complications. The dosage is likely to depend on such variables as the type and extent of progression of the COVID-19, the severity of the COVID-19, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the COVID-19 or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of COVID-19.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

References: The following documents are incorporated into the present disclosure as if fully restated herein: Magalhães G S, Rodrigues-Machado M G, Motta-Santos D, Silva A R, Caliari M V, Prata L O, Abreu S C, Rocco P R, Barcelos L S, Santos R A, Campagnole-Santos M J. Angiotensin-(1-7) attenuates airway remodelling and hyper responsiveness in a model of chronic allergic lung inflammation. Br J Pharmacol. 2015 May; 172(9):2330-42. doi: 10.1111/bph.13057. Epub 2015 Mar. 18. PubMed PMID: 25559763; PubMed Central PMCID: PMC4403097; Li N, Cai R, Niu Y, Shen B, Xu J, Cheng Y. Inhibition of angiotensin II-induced contraction of human airway smooth muscle cells by angiotensin-(1-7) via downregulation of the RhoA/ROCK2 signaling pathway. Int J Mol Med. 2012 October; 30(4):811-8. doi: 10.3892/ijmm.2012.1080. Epub 2012 Jul. 27. PubMed PMID: 22842919; Hemnes A R, Rathinasabapathy A, Austin E A, Brittain E L, Carrier E J, Chen X, Fessel J P, Fike C D, Fong P, Fortune N, Gerszten R E, Johnson J A, Kaplowitz M, Newman J H, Piana R, Pugh M E, Rice T W, Robbins I M, Wheeler L, Yu C, Loyd J E, West J. A potential therapeutic role for angiotensin-converting enzyme 2 in human pulmonary arterial hypertension. EurRespir J. 2018 Jun. 21; 51(6). pii: 1702638. doi: 10.1183/13993003.02638-2017. Print 2018 Jun. PubMed PMID: 29903860; PubMed Central PMCID: PMC6613216. Tan WSD, Liao W, Zhou S, Mei D, Wong W F. Targeting the renin-angiotensin system as novel therapeutic strategy for pulmonary diseases. Curr Opin Pharmacol. 2018 June; 40:9-17. doi: 10.1016/j.coph.2017.12.002. Epub 2017 Dec. 27. Review. PubMed PMID: 29288933; Deshotels M R, Xia H, Sriramula S, Lazartigues E, Filipeanu C M. Angiotensin II mediates angiotensin converting enzyme type 2 internalization and degradation through an angiotensin II type I receptor-dependent mechanism. Hypertension. 2014 December; 64(6):1368-1375. doi: 10.1161/HYPERTENSIONAHA.114.03743. Epub 2014 Sep. 15. Erratum in: Hypertension. 2014 December; 64(6):e8. Sriramula, Srinivas [added]. PMID: 25225202; PMCID: PMC4231883.

Wherefore, We claim:

1. A method of treating COVID-19 in a patient in need thereof comprising
administering a pharmaceutical composition containing a pharmaceutically effective dose of a therapeutic,
wherein the therapeutic contains both an ACE2 externalizer and one or more ACE2 internalization preventors.

2. The method of claim 1 wherein the ACE2 externalizer is one of diminazene, diminazene aceturate, or a pharmaceutically acceptable salt, solvate, ester, amide, clathrate, stereoisomer, enantiomer, prodrug or analog thereof.

3. The method of claim 1 wherein the one or more ACE2 internalization preventor is one or more of a cholesterol binding drug, a cholesterol synthesis inhibitor, and an angiotensin type 1 receptor antagonist.

4. The method of claim 3, wherein the cholesterol binding drug is ezetimibe or a pharmaceutically acceptable salt, solvate, ester, amide, clathrate, stereoisomer, enantiomer, prodrug or analog thereof.

5. The method of claim 3 wherein the cholesterol synthesis inhibitor is one of atorvastati, cerivastatin, fluvastatin, lovastatin, mevastatin, pitavastatin, pravastatin, rosuvastatin, and simvastatin, or a pharmaceutically acceptable salt, solvate, ester, amide, clathrate, stereoisomer, enantiomer, prodrug or analog thereof.

6. The method of claim 3 wherein the angiotensin type 1 receptor antagonist is one of azilsartan, candesartan, eprosartan, irbesartan, telmisartan, valsartan, losartan, olmesartan, entresto, byvalson and fimasartan or a pharmaceutically acceptable salt, solvate, ester, amide, clathrate, stereoisomer, enantiomer, prodrug or analog thereof.

7. The method of claim 3 wherein the one or more ACE2 internalization preventor includes at least one of each of a cholesterol binding drug, a cholesterol synthesis inhibitor, and an angiotensin type 1 receptor antagonist.

8. A pharmaceutical composition comprising
an ACE2 externalizer; and
one or more ACE2 internalization preventors.

9. The pharmaceutical composition of claim 8, wherein the ACE2 externalizer is one of diminazene, diminazene aceturate, or a pharmaceutically acceptable salt, solvate, ester, amide, clathrate, stereoisomer, enantiomer, prodrug or analog thereof.

10. The pharmaceutical composition of claim 8, wherein the one or more ACE2 internalization preventor is one or more of a cholesterol binding drug, a cholesterol synthesis inhibitor, and an angiotensin type 1 receptor antagonist.

11. The pharmaceutical composition of claim 8, wherein the cholesterol binding drug is ezetimibe or a pharmaceutically acceptable salt, solvate, ester, amide, clathrate, stereoisomer, enantiomer, prodrug or analog thereof.

12. The pharmaceutical composition of claim 11, wherein the cholesterol synthesis inhibitor is one of atorvastati, cerivastatin, fluvastatin, lovastatin, mevastatin, pitavastatin, pravastatin, rosuvastatin, and simvastatin, or a pharmaceutically acceptable salt, solvate, ester, amide, clathrate, stereoisomer, enantiomer, prodrug or analog thereof.

13. The pharmaceutical composition of claim 11, wherein the angiotensin type 1 receptor antagonist is one of azilsartan, candesartan, eprosartan, irbesartan, telmisartan, valsartan, losartan, olmesartan, entresto, byvalson and fimasartan or a pharmaceutically acceptable salt, solvate, ester, amide, clathrate, stereoisomer, enantiomer, prodrug or analog thereof.

14. The pharmaceutical composition of claim 11, wherein the one or more ACE2 internalization preventor includes at least one of each of a cholesterol binding drug, a cholesterol synthesis inhibitor, and an angiotensin type 1 receptor antagonist.

15. A method of treating COVID-19 in a patient in need thereof comprising
administering a pharmaceutically effective dose of a therapeutic,
wherein the therapeutic contains an ACE2 externalizer.

16. The method of claim 15, wherein the ACE2 externalizer is one of diminazene, diminazene aceturate, or a pharmaceutically acceptable salt, solvate, ester, amide, clathrate, stereoisomer, enantiomer, prodrug or analog thereof.
